(19) United States
(12) Patent Application Publication
(10) Pub. No.: US 2007/0015989 A1
(43) Pub. Date: Jan. 18, 2007

(54) Endoscope Image Recognition System and Method

(75) Inventors: Rupesh Desai, San Jose, CA (US); Fred R. Seddiqui, Los Altos, CA (US); Jack Higgins, Los Altos, CA (US); Alex C. Niel, Daly City, CA (US)

Correspondence Address: SQUIRE, SANDERS & DEMPSEY L.L.P., 1 MARITIME PLAZA, SUITE 300, SAN FRANCISCO, CA, 94111, US

(73) Assignee: AVANTIS MEDICAL SYSTEMS, INC., Sunnyvale, CA (US)

(21) Appl. No.: 11/160,646

(22) Filed: Jul. 1, 2005

(51) Int. Cl. A61B 5/05

(52) U.S. Cl. 600/407

(57) ABSTRACT

An endoscope is communicatively coupled to electronics that receive images of tissue from the endoscope and determine a correlation between the received images and images of normal and abnormal tissues in a database. Based on the determined correlation, an operator of the endoscope is notified of the correlation and the electronics can cause the endoscope to act accordingly (e.g., biopsy abnormal tissue).

### TECHNICAL FIELD

The present invention relates to implantable medical devices for capturing images. More specifically, the present invention relates to an endoscope system having a visual element communicatively coupled to an image recognition system for differentiating normal from abnormal tissue.

### BACKGROUND

An endoscope is a medical device comprising a camera mounted on a flexible tube. Small instruments can be used to take samples of suspicious tissues or to perform other surgical procedures through the endoscope. For example, gastroscopes are used for esophagus, stomach, duodenum; colonoscopes for examination of colon; bronchoscopes for the bronchi; laparoscopes for peritoneal cavity; sigmoidoscopes for the rectum and the sigmoid colon; and angioscopes for the examination of blood vessels.

With the use of endoscopes with all of these procedures, the commonality is the use of a camera to assist the health care provider in directing the endoscope as well as looking for abnormalities that are to be treated. Endoscopes are designed either with a single camera attached to the distal end of the flexible tube or with a fiberoptic bundle that transmits an image from a lens at the distal end of the scope to an eyepiece or video camera at the proximal end. Accordingly, a scope provides for a two dimensional visual feedback from the prospective of the position of the end of the scope.

When a potential abnormality is viewed with the endoscope, an examiner must decide whether to perform a biopsy (take a sample for later microscopic examination in a clinical laboratory) or to attempt complete removal of the abnormality (e.g., perform a polypectomy to remove a polyp in the colon). However, attempting to remove the abnormality adds significant risks of complications due to the increased possibility of perforation of the surrounding tissue (e.g., the bowel wall in a polypectomy). Accordingly, it is important to avoid unnecessary tissue removals but it is also important to avoid missing or ignoring abnormal tissues, thus exposing a patient to the possibility that cancerous or pre-cancerous tissue may have been left undetected.

Although it is sometimes easy for a physician to determine whether it is appropriate to biopsy or completely remove abnormal tissue, it is often difficult for an experienced physician to predict the microscopic diagnosis based on visual examination of the surface of the abnormal tissue. This is true for when the physician is onsite as well as when the physician is located at a remote site and operating the endoscope via a robotic mechanism.

It is sometimes possible for the physician to obtain guidance by ordering an immediate pathological examination of a biopsy specimen, thereby enabling an immediate decision of whether to remove abnormal tissue. However, this is expensive and cannot always be arranged on short notice, even when the procedure is performed in a hospital or surgery center. It is even more difficult and expensive to order a pathological examination in a less sophisticated facility having a limited or no laboratory, as is often the case when the procedure is performed via remote operation.

Accordingly, a new system and method are needed that can accurately, quickly and inexpensively provide guidance to a physician during an endoscope procedure.

### SUMMARY

Embodiments of the invention provide an apparatus and method for imaging tissue within a body and determining whether the imaged tissue is abnormal.

In one embodiment the apparatus comprises a sensor communicatively coupled to electronics. The electronics comprise a sensor engine, an image engine, and an alarm engine. The sensor is capable of travel within a body and of imaging tissue within the body. The sensor engine is capable of receiving an image of tissue from the sensor. The image engine is capable of determining a correlation between the received image and images of normal and abnormal tissues in a database. The alarm engine is capable of outputting information to an operator of the apparatus based on the determination such that the operator can act accordingly.

In an embodiment of the invention, the method comprises: receiving an image of tissue from a sensor capable of travel within a body; determining a correlation between the received image and images of normal and abnormal tissues in a database; and outputting information to an operator of an apparatus based on the determination.

### DESCRIPTION OF THE FIGURES

Non-limiting and non-exhaustive embodiments of the present invention are described with reference to the following figures, wherein like reference numerals refer to like parts throughout the various views unless otherwise specified.

FIG. 1 is a diagram illustrating an endoscope image matching system according to an embodiment of the invention;

FIG. 2 is a diagram illustrating a computer of the system of FIG. 1;

FIG. 3 is a diagram illustrating an input section of an input/output interface of the computer of FIG. 1;

FIG. 4 is a diagram illustrating a persistent memory of the computer of FIG. 1;

FIG. 5 is a diagram illustrating a database of the persistent memory of FIG. 4; and

FIG. 6 is a flowchart illustrating a method of operating the endoscope image matching system of FIG. 1.

### DETAILED DESCRIPTION OF THE ILLUSTRATED EMBODIMENTS

The following description is provided to enable any person having ordinary skill in the art to make and use the invention, and is provided in the context of a particular application and its requirements. Various modifications to the embodiments will be readily apparent to those skilled in the art, and the principles defined herein may be applied to other embodiments and applications without departing from the spirit and scope of the invention. Thus, the present invention is not intended to be limited to the embodiments shown, but is to be accorded the widest scope consistent with the principles, features and teachings disclosed herein.

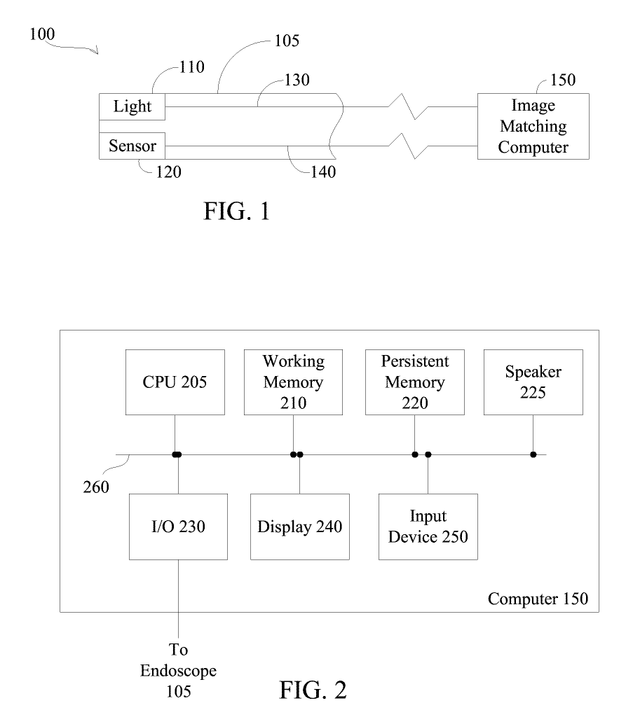

FIG. 1 is a diagram illustrating an endoscope image matching system 100 according to an embodiment of the invention. The system 100 comprises an endoscope 105 communicatively coupled to an image matching computer 150. The endoscope 105, as is known by one of ordinary skill in the art, can be inserted into the body for examination and imaging of tissue therein. The computer 150, as will be discussed in further detail below, analyzes tissue imaged by the endoscope 105. The endoscope 105 includes a light 110 and a sensor 120. The light 110, which can include a light emitting diode or fiber optic bundle in one embodiment, emits light from the endoscope 105 thereby illuminating tissue. The light 110 can emit light in different spectrums, including infrared, visual and ultraviolet. The sensor 120, which can include a Complementary Metal-Oxide Semiconductor (CMOS), Charge-Coupled Device (CCD), a fiber optic bundle, and/or other imaging devices, images the illuminated tissue and transmits the images to the computer 150 for analysis via a wired connection 140. The light 110 can also be communicatively coupled to the computer 150 via a wired connection 130. In an embodiment of the invention, the light 110 and/or the sensor 120 are wirelessly connected, e.g., via Ultra Wideband, WiFi, etc., to the computer 150.

It will be appreciated by one of ordinary skill in the art that the endoscope 105 can include additional components, such as a gas channel for injecting a gas, such CO2, into the body, a retractable needle for drug injection, hydraulically actuated scissors, clamps, grasping tools, electrocoagulation systems, ultrasound transducers, electrical sensors, heating elements, other ablation devices, etc. Further, the endoscope 105 can also include a surgical apparatus (e.g., a snare) remotely controlled by a physician for performing biopsies and other tissue removal processes.

In an embodiment of the invention, the computer 150 may be miniaturized and integrated with the sensor 120 such that the endoscope 105 includes the computer 150, thereby eliminating the need to transfer data external to the body. In another embodiment of the invention, the computer 150 can be communicatively coupled to any other device for imaging tissue within the body, such as the Given Imaging PILLCAM Capsule Endoscopy system.

In an embodiment of the invention, the endoscope 105 includes a self-propelling endoscope, as is known in the art, which can advance when instructed to by the computer 150. In another embodiment, the sensor 120 includes an ultrasound transducer and receiver for emitting ultrasound and imaging tissue based on the emitted ultrasound.

FIG. 2 is a diagram illustrating the computer 150 of the system 100 (FIG. 1). The computer 150 includes a central processing unit (CPU) 205; working memory 210; persistent memory 220; a speaker 225; input/output (I/O) interface 230; display 240; and input device 250, all communicatively coupled to each other via a bus 260. The CPU 205 may include an INTEL PENTIUM microprocessor, a Motorola POWERPC microprocessor, or any other processor capable to execute software stored in the persistent memory 220. The working memory 210 may include random access memory (RAM) or any other type of read/write memory devices or combination of memory devices. The persistent memory 220 may include a hard drive, read only memory (ROM) or any other type of memory device or combination of memory devices that can retain data after the computer 150 is shut off. The persistent memory 220 will be discussed in further detail below. The speaker 225 is capable of outputting audio according to the software stored in the persistent memory 220. The I/O interface 230 is communicatively coupled, via wired or wireless techniques, to the light 110 and/or the sensor 120. The display 240 may include a flat panel display, cathode ray tube display, or any other display device. The input device 250, which is optional like other components of the invention, may include a keyboard, mouse, or other device for inputting data, or a combination of devices for inputting data.

One skilled in the art will recognize that the computer 150 may also include additional devices, such as network connections, additional memory, additional processors, LANs, input/output lines for transferring information across a hardware channel, the Internet or an intranet, etc. One skilled in the art will also recognize that the programs and data may be received by and stored in the system in alternative ways. Further, in an embodiment of the invention, an Application Specific Integrated Circuit (ASIC) is used in placed of the computer 150.

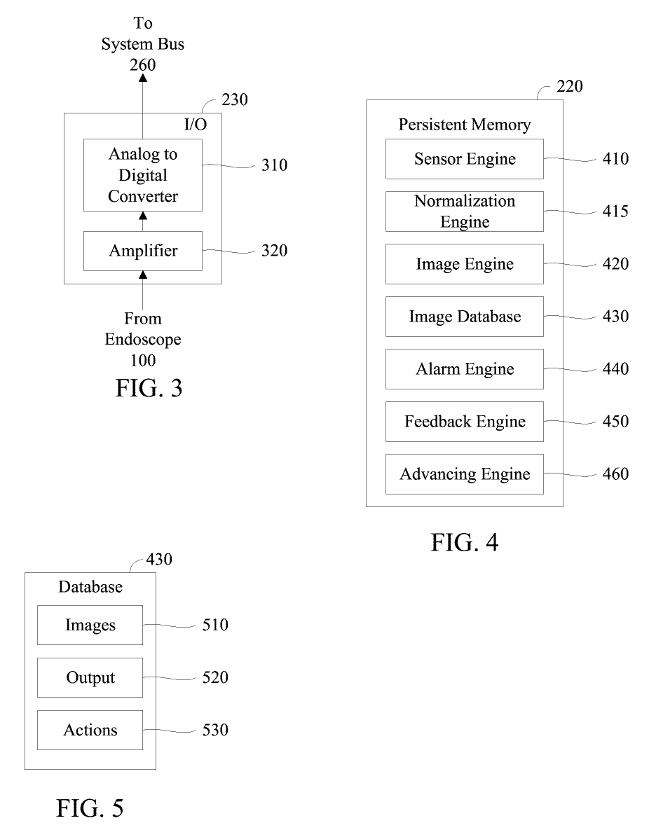

FIG. 3 is a diagram illustrating an input section of the input/output interface 230 of the computer 150 (FIG. 1). The I/O interface 230 is communicatively coupled to the light 110 and/or the sensor 120 and receives data from the sensor 120. The input section includes an amplifier 320 and an analog to digital converter (ADC) 310. If the data from sensor 120 is in analog format, then the amplifier 320 amplifies the data and then the ADC 310 converts the analog data to digital data for processing by the computer 150. If the data from the sensor 120 is in digital format, then amplification by the amplifier 320 and conversion by the ADC 310 are not needed.

FIG. 4 is a diagram illustrating the persistent memory 220 of the computer 150 (FIG. 1). The persistent memory 220 includes a sensor engine 410, a normalization engine 415, an image engine 420, an image database 430, an alarm engine 440, a feedback engine 450, and an advancing engine 460. The sensor engine 410 receives data from the sensor 120 and converts into a format understandable by the image engine 420. The sensor engine 410 also causes the light 110 to emit light at different wavelengths so that the suspect tissue can be imaged at different wavelengths. The normalization engine 415 normalizes the received data. Normalization includes image size and/or image intensity due to differences in range between the sensor 120 and the tissue and differences in sensors 120 (due to variations in manufacturing processes of the sensors 120 and to light conditions), respectively. The normalization engine 415 can also normalize received data to match image size, intensity, color, etc. of images in the database 430. The image engine 420 analyzes the received normalized data and matches the received normalized data with images stored in the image database 430, as will be discussed further below. The image database 430 includes images 510, associated output 520 and associated actions 530, as will be discussed in further detail below.

The alarm engine 440 sounds an alarm, aurally on the speaker 225 and/or visually on the display 240 when the image engine 420 determines the probability of a match exceeds a certain threshold based on a correlation between imaged tissue and tissue in the database 430, as well other factors in some embodiments (e.g., age and/or ethnicity of the patient). The alarm engine 440 alerts the physician by displaying on the display 240 and/or reading out on the speaker 225 the imaged tissue, the matched tissue(s) from the images 510, the probability of a match or matches, the identity of the images (e.g., cancerous tissue, benign tumor, etc.), suggested actions and/or other data. The feedback engine 450 takes actions or causes the endoscope 105 to take actions stored in the actions 510 that are associated with the match if automatic actions are enabled. For example, for a cancerous tissue match, the associated action would be removal of the tissue, for which the feedback engine 450 would cause the endoscope 105 to remove the tissue imaged by the sensor 120. The advancing engine 460 advances the endoscope 105 when appropriate based on results from the image engine 420 (e.g., can advance without other actions when tissue is identified as non-cancerous by the image engine 420).

The image engine 420 applies various algorithms to compare the data received from the sensor 120 with images stored in the image database 430. In an embodiment of the invention, an algorithm used can determine the correlation, p, between images, e.g., between sensor 120 images and images stored in the database 430. For example:

$$ρ = \frac{{\sum }_{r = 1}^{R} {\sum }_{c = 1}^{C} ( {g}_{1} ( r , c ) - {μ}_{1} ) ( {g}_{2} ( r , c ) - {μ}_{2} )}{\sqrt{{\sum }_{r = 1}^{R} {\sum }_{c = 1}^{C} {( {g}_{1} ( r , c ) - {μ}_{1} )}^{2} {\sum }_{r = 1}^{R} {\sum }_{c = 1}^{C} {( {g}_{2} ( r , c ) - {μ}_{2} )}^{2}}} ; - 1 \leq ρ \leq 1$$

wherein

g₁(r,c)=individual gray values of sensor image

μ₁=average gray value of sensor image

g₂(r,c)=individual gray values of corresponding part of database image

μ₂=average gray value of corresponding part of database image

R,C=number of rows and columns of sensor image.

A higher correlation value indicates the higher likelihood of a match between the tissue imaged by the sensor 120 and the tissue image in the database 430. In another embodiment of the invention, the image engine 420 looks for and matches colors of tissue that indicate diseased tissue. For example, white coloration of tissue within the bowel may indicate inflammatory bowel disease or a similar condition (e.g., Crohn's Disease). The corresponding action would be either biopsy or no action. In an embodiment of the invention, the image engine 420 looks for a change in color of tissue to indicate when to do an image matching analysis of imaged tissue.

FIG. 5 is a diagram illustrating the image database 430 of the persistent memory 220 (FIG. 4). The image database 430 includes records for images 510, corresponding output 520, and corresponding actions 530. The images 510 includes images of different types of diseased and other abnormal tissue as well as images of variations of normal tissue. The images 510 can include identical tissue imaged at different wavelengths and/or in ultrasound. For each image in images 510, there is a corresponding output in output 520 which includes the identity of the abnormal tissue, characteristics of the abnormal tissue (e.g., color, size, etc.), and other data. For each image in images 510, there is also a corresponding action or actions in the actions 530. Actions in action 530 can include tissue removal, biopsy, advancing the endoscope, etc. Actions 530 also indicates at what correlation or probability should the action be taken. For example, a biopsy, which has less risk than full tissue removal, could require a lower correlation/probability of a match for the action to be taken than would the full tissue removal.

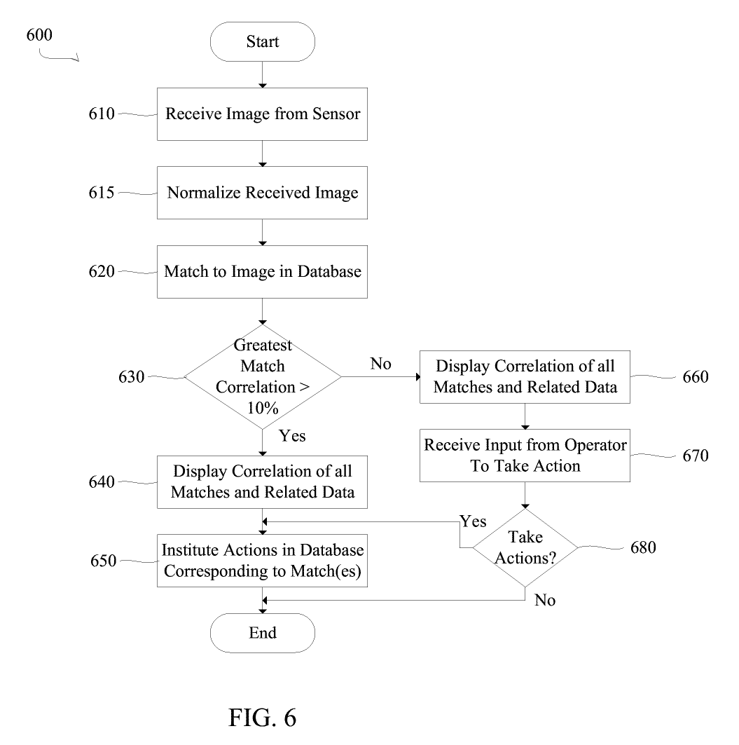

FIG. 6 is a flowchart illustrating a method 600 of operating the endoscope image matching system 100. In an embodiment of the invention, the system 100 executes the method 600. First, an image is received (610) from the sensor 120. The image is the normalized (615) to correct for different image sizes (e.g., due to different ranges from which the tissue is imaged), intensity (e.g., due to variations in lighting and or sensors) and/or other factors. Then the received normalized image is matched (620) to one or more images in the database 430. If (630) there is at least one match with a correlation of greater than, for example, 10%, then the correlation of the match or matches is displayed (640) along with related data, such as identity of the matches and recommended actions. The corresponding actions are then instituted (650). In an embodiment of the invention, the corresponding actions are instituted (650) if the correlation is greater than a certain percentage for that match. The method 600 then ends. If (630) there is no match greater than 10%, then the correlations of all matches are displayed (660) with related data with a prompt to take action or not. Input is then received (670) to take action or not. If (680) action is to be taken, then actions indicated in the database 430 are instituted (650) and the method 600 ends. Otherwise, the method 600 ends.

In an embodiment of the invention, the calculated correlation can be adjusted based on demographic factors related to the patient being examined. For example, an older patient may have a higher chance of colorectal cancer than a younger patient and therefore the correlation would be increased for an older patient and similarly decreased for a younger patent. In another example, Japanese have a higher likelihood of getting colorectal cancer and therefore the correlation can be increased for Japanese patients and decreased for European patients.

The foregoing description of the illustrated embodiments of the present invention is by way of example only, and other variations and modifications of the above-described embodiments and methods are possible in light of the foregoing teaching. Although the engines are being described as separate and distinct, one skilled in the art will recognize that these engines may be a part of an integral site, may each include portions of multiple engines, or may include combinations of single and multiple engines. Further, components of this invention may be implemented using a programmed general purpose digital computer, using application specific integrated circuits, or using a network of interconnected conventional components and circuits. Connections may be wired, wireless, modem, etc. The embodiments described herein are not intended to be exhaustive or limiting. The present invention is limited only by the following claims.

What is claimed is:

1. An apparatus, comprising:
a sensor capable of travel within a body and of imaging tissue within the body; and
electronics, communicatively coupled to the sensor, comprising
a sensor engine capable of receiving an image of tissue from the sensor,
an image engine capable of determining a correlation between the received image and an image of abnormal tissue in a database, and
an alarm engine capable of outputting information to an operator of the apparatus based on the determination.

2. The apparatus of claim 1, further comprising a surgical device capable of removing imaged tissue based on the determination.

3. The apparatus of claim 1, wherein the output includes an identification of abnormal tissue from the database that has a correlation with the imaged tissue, the correlation with the abnormal tissue, and corresponding proposed actions.

4. The apparatus of claim 1, further comprising a light, communicatively coupled to the electronics, capable of illuminating the imaged tissue at different wavelengths.

5. The apparatus of claim 1, wherein the correlation is adjusted for demographic factors of a patient.

6. The apparatus of claim 1, further comprising a surgical apparatus communicatively coupled to the electronics and wherein the electronics further comprises a feedback engine capable to cause the surgical apparatus to perform an action based on the determination.

7. The apparatus of claim 1, further comprising an advancing mechanism communicatively coupled to the electronics and coupled to the sensor and wherein the electronics further comprises an advancing engine capable of causing the advancing mechanism to advance the sensor based on the determination.

8. The apparatus of claim 1, wherein the alarm engine aurally and visually outputs the information.

9. The apparatus of claim 1, wherein the image engine performs the determination upon detecting a change in tissue color.

10. The apparatus of claim 1, wherein the electronics further comprises a normalization engine capable of normalizing the received image to the abnormal tissue image in the database.

11. The apparatus of claim 10, wherein the normalizing adjusts the size of the received image.

12. A method, comprising:
receiving, by electronics, an image of tissue from a sensor capable of travel within a body;
determining, with the electronics, a correlation between the received image and an image of abnormal tissue in a database; and
outputting, by the electronics, information to an operator of an apparatus based on the determination.

13. The method of claim 12, further comprising causing, by electronics, removing, with a surgical device, the imaged tissue based on the determination.

14. The method of claim 12, wherein the output includes an identification of abnormal tissue from the database that has a correlation with the imaged tissue, the correlation with the abnormal tissue, and corresponding proposed actions.

15. The method of claim 12, further comprising illuminating the imaged tissue at different wavelengths.

16. The method of claim 12, wherein the correlation is adjusted for demographic factors of a patient.

17. The method of claim 12, further comprising causing, by the electronics, the sensor to advance based on the determination.

18. The method of claim 12, wherein the information is output visually and aurally.

19. The method of claim 12, further comprising detecting a change in color of imaged tissue and performing the determining when there is a detected change.

20. The method of claim 12, further comprising normalizing the received image to the abnormal tissue image in the database.

21. The method of claim 20, wherein the normalizing adjusts the size of the received image.

22. A computer-readable medium having stored thereon instructions to cause a computer to execute a method, the method comprising:
receiving an image of tissue from a sensor capable of travel within a body;
determining a correlation between the received image and an image of abnormal tissue in a database; and
outputting information to an operator of an apparatus based on the determination.

23. A system, comprising:
means for receiving an image of tissue from a sensor capable of travel within a body;
means for determining a correlation between the received image and an image of abnormal tissue in a database; and
means for outputting information to an operator of an apparatus based on the determination.
